(19) United States
(12) Patent Application Publication
(10) Pub. No.: US 2023/0265513 A1
(43) Pub. Date: Aug. 24, 2023

(54) METHODS FOR THE DETECTION OF LENGTH POLYMORPHISMS

(71) Applicant: VIRGINIA COMMONWEALTH UNIVERSITY, RIchmond, VA (US)

(72) Inventors: Jason REED, Richmond, VA (US); Sean Koebley, Richmond, VA (US)

(21) Appl. No.: 18/003,949

(22) PCT Filed: Jun. 30, 2021
(86) PCT No.: PCT/US2021/039746

§ 371 Date: Dec. 30, 2022

Related U.S. Application Data
(60) Provisional application No. 63/045,877, filed on Jun. 30, 2020.

(51) Int. Cl. C12Q 1/6883; C12Q 1/6858

(52) U.S. Cl. CPC C12Q 1/6883; C12Q 1/6858; C12Q 2600/156

(57) ABSTRACT

Methods for detecting length polymorphisms in a DNA sample are provided. The methods include steps of amplifying a target region of the DNA in the sample, wherein the DNA sample is diluted prior to the amplifying step to provide a plurality of amplified samples in which 0 or 1 target DNA strand is amplified in each amplified sample and wherein at least one amplified sample includes 1 target DNA strand; depositing the plurality of amplified samples onto a surface; imaging the plurality of amplified samples deposited on the surface using atomic force microscopy (AFM) to determine an amplicon length distribution; comparing the amplicon length distribution to a corresponding amplicon length distribution obtained from a reference DNA sample that does not contain a length polymorphism in the target region; and detecting a length polymorphism in the target region of the DNA sample when the amplicon length distribution is distinct from the corresponding amplicon length distribution.

### CROSS-REFERENCE TO RELATED APPLICATIONS

This application claims priority to U.S. Provisional Application 63/045,877 filed on Jun. 30, 2020. The complete content thereof is herein incorporated by reference.

### FIELD OF THE INVENTION

The invention is generally related to methods of detecting length polymorphisms by amplifying a target genomic region and imaging the amplified region using atomic force microscopy.

### BACKGROUND OF THE INVENTION

Length polymorphisms associated with genetic disease are currently among the most difficult variant types to characterize, and their clinical impact is significant. Nucleotide repeat expansions are found in individuals with Fragile X syndrome, Huntington disease, and most hereditary ataxias,^{1,2} while copy number variations and tandem duplications are associated with lung,³ breast,^{4,5} blood,⁶ and prostate⁷ cancers. In these cases, the insertion or deletion is of variable length, composed to some degree of a repeated sequence, and often longer than the short reads used in next-generation sequencing (NGS)—challenges that limit the detection of variants and accurate reporting of indel length by traditional assays like microarrays and targeted sequencing.¹

For example, FLT3 internal tandem duplications (ITDs), identified in about 30% of AML cases, are associated with a poor prognosis, especially if the variant allele frequency (VAF) is greater than 50%.^{6,9-11} In WT patients, the FLT3 gene expresses an fms-like tyrosine kinase receptor that is involved in cell growth and division pathways.¹² FLT3-ITDs are associated with overactivation of the pathway, yielding uncontrolled cellular proliferation and formation of myeloid blasts characteristic of AML. Most detected ITDs are reported to be less than 100 bp in length, with a mode length of about 40 bp.^{13,14} Since the expression level of the FLT3-ITD, mutation size, and insertion location are linked to survivability^{6,9,11,15,16} and determine sensitivity to different drugs,^{9,12} quantification of variant length and frequency are of urgent concern for informed AML treatment.

Existing assays to detect FLT3-ITDs present shortcomings in throughput, accuracy, or feasibility. In PCR-CE techniques, e.g. the widely used commercial Leukostrat CDx FLT3 Mutation Assay, PCR is used to amplify the 368 bp ITD region within the FLT3 gene, capillary gel electrophoresis (CE) gives the length of the resulting amplicons, and amplicon length indicates the presence and length of an insertion.¹³ However, due to PCR amplification bias and inherent limitations of CE, the lowest VAF that can be detected is 5%, the sensitivity to longer variants is even more limited, and, crucially, the VAF is not reported due to lack of reliability.^{13,17} Multi-parameter flow cytometry can detect AML-associated combinations of cell antigens at VAFs of 10⁻³ to 10⁻⁴, but the genetic character of a mutation is not directly identified, and the technique requires live cells and extensive expertise.¹⁷ Denaturing high performance liquid chromatography can detect insertion sites and lengths at VAF>0.025,^{11,18,19} but its expense and length limitations narrows its appeal. Karyotyping lacks sufficient resolution to effectively detect FLT3-ITDs.⁶ Quantitative PCR (qPCR) is a reliable and cost-effective approach that can detect many AML-associated chromosomal rearrangements and mutations, but the lack of a consistent target in length polymorphisms like FLT3-ITDs is a major barrier, as is the attenuation of variant signal due to amplification bias in favor of a shorter WT target.^{8,17} NGS is a strong candidate for characterizing FLT3-ITDs: FLT3 is included amongst the targeted genes in major NGS myeloid panels,²⁰ and minimum residual disease detection of FLT3-ITDs at extremely low (<10⁻⁵) variant fractions was recently demonstrated.^{15,20} However, the feasibility of NGS for comprehensive and accurate FLT3-ITD reporting remains a concern; notably, (a) repetitive inserts of variable length, especially those longer than the NGS reads, present a significant challenge,²⁰ and (b) the cost and time required for multiple specialized NGS assays over the course of a single patient's treatment would necessitate considerable additional expense. Single-molecule sequencing platforms may eventually overcome the repeatability and length limitations of NGS, but cost and reliability remain issues for these emergent techniques for the foreseeable future.²¹ To improve upon the often-serious health outcomes of patients with length polymorphisms, there is a need for a rapid, low-cost, and accurate assay that can meet the challenges of length heterogeneity and nucleotide repetition.

### SUMMARY

Described herein are methods for determining the length and frequency of a length polymorphism which combines amplification techniques such as digital polymerase chain reaction (dPCR) followed by single-molecule length measurement using atomic force microscopy (AFM).

An aspect of the disclosure provides a method for detecting length polymorphisms in a DNA sample, comprising amplifying a target region of the DNA in the sample, wherein the DNA sample is diluted prior to the amplifying step to provide a plurality of amplified samples in which 0 or 1 target DNA strand is amplified in each amplified sample and wherein at least one amplified sample includes 1 target DNA strand; depositing the plurality of amplified samples onto a surface; imaging the plurality of amplified samples deposited on the surface using AFM to determine an amplicon length distribution; comparing the amplicon length distribution to a corresponding amplicon length distribution obtained from a reference DNA sample that does not contain a length polymorphism in the target region; and detecting a length polymorphism in the target region of the DNA sample when the amplicon length distribution is distinct from the corresponding amplicon length distribution.

In some embodiments, the amplifying step is performed using dPCR. In some embodiments, the DNA sample and the reference DNA sample are combined into a single sample before the amplifying step in a 1:1 ratio. In some embodiments, the method further comprises a step of determining a percentage frequency of the length polymorphism in the DNA sample as compared to the reference DNA sample. In some embodiments, a Bayesian statistical analysis is used to determine whether the amplicon length distribution is distinct from the corresponding amplicon length distribution of the reference DNA sample.

In some embodiments, the AFM is high-speed AFM. In some embodiments, the amplicon length distribution is determined by a computer-implemented method comprising the steps of flattening an AFM image; thresholding to identify DNA strands; skeletonizing the thresholded strands; determining the longest backbone of each skeleton; measuring the length of the longest backbone; and applying a quality filter. In some embodiments, the length polymorphism is an internal tandem duplication. In some embodiments, the target region is labeled with CRISPR associated protein 9 (Cas9).

### BRIEF DESCRIPTION OF THE DRAWINGS

FIG. 1. Overview of dPCR-HSAFM for the quantification of length polymorphisms in a heterogeneous sample. In the dPCR step, a starting mixed sample was diluted, aliquoted into individual wells of a PCR plate, and amplified, resulting in homogeneous amplicons within each well. For HSAFM scanning, each well's contents were deposited onto a subdivided mica grid and imaged with high-speed atomic force microscopy at nanoscale resolution. The strands were traced by custom software to yield length distributions for each well, and those distributions were analyzed with a Bayesian approach to determine if they were most likely WT or variant in origin.

FIGS. 2A-F. (A,B) Example HSAFM scans from well 8 of W-0 and well 13 of M-100 (+30 bp insert), (C,D) strand length distributions from wells 1-20 of W-0 and wells 1-20 of M-100, (E) ROC curve displaying decision test results of WT and M-100 for various ROPEs, and (F) all W-0 and M-100 95% HDIs shown relative to the optimal ROPE. In (A) and (B), example scan images and traced amplicons are shown for the two example wells. For each well, 55 images were scanned and traced to produce length distributions. In (C) and (D), each violin plot shows the length distribution from a single well, and horizontal dashed lines show the optimal ROPE found by the ROC curve (115.8-131.0 nm). The vertical bar within each violin plot gives the well's 95% HDI (95% most credible mean lengths as calculated by Bayesian posteriors). In (E), the solid line gives the false positive rate (FPR) and true positive rate (TPR) for each tested ROPE, and the “x” shows the optimal ROPE. The area under the ROC curve (AUC) is 0.988. In (F), dotted lines show the optimal ROPE. Vertical lines show 95% HDIs for all wells in W-0 and M-100.

FIGS. 3A-B. (A) 95% HDIs and (B) variant allele fractions for all samples. In (A), numbers give the number of non-excluded wells for each sample, vertical lines are 95% most credible mean lengths (HDIs) for all wells as calculated from Bayesian posteriors, dotted lines are ROPE bounds, shaded regions surrounding the dotted lines give the ROPE bounds ±10% of the ROPE width. In (B), solid horizontal lines give the variant allele fractions (VAFs) as determined by the decision test using the ROPE, and shaded bars give the range of VAFs as determined by the decision test with ROPE ±10% of the ROPE width. Example decision test results and resulting VAF calculation are shown for sample M-100.

FIG. 4. Spiked-in VAF v. detected VAF for MV4-11 and PL-21 samples, plotted on a log-log scale. Markers give the VAF for each sample as determined by decision test results using the ROPE, and vertical bars give the range of VAFs as determined by the decision test with ROPE ±10% of the ROPE width.

### DETAILED DESCRIPTION

Embodiments of the disclosure provide the use of atomic force microscopy (AFM) in combination with DNA amplification techniques to detect length polymorphisms. The term “polymorphism”, as used herein, refers to the coexistence of more than one form of a gene or portion thereof.

Exemplary amplification techniques include polymerase chain reaction (PCR), including digital PCR (dPCR), droplet digital PCR (ddPCR), ligase chain reaction, antisense RNA amplification, NASBA, etc. In contrast to conventional PCR in which one reaction is performed per well, dPCR involves partitioning the PCR solution into tens of thousands of nano-liter sized droplets, where a separate PCR reaction takes place in each one. In methods of the present disclosure, the sample is diluted so that one target molecule is present in a single PCR reaction. This allows the determination of how many different length target molecules are in the sample. A PCR solution is made similarly to a TaqMan assay, which consists of template DNA (or RNA), fluorescence-quencher probes, primers, and a PCR master mix, which contains DNA polymerase, dNTPs, MgCl₂, and reaction buffers at optimal concentrations. Several different methods can be used to partition samples, including microwell plates, capillaries, oil emulsion, and arrays of miniaturized chambers with nucleic acid binding surfaces. The PCR solution is divided into smaller reactions and are then made to run PCR individually. After multiple PCR amplification cycles, the samples are checked for fluorescence with a binary readout of “0” or “1”. The fraction of fluorescing droplets is recorded. The partitioning of the sample allows one to estimate the number of different molecules by assuming that the molecule population follows the Poisson distribution, thus accounting for the possibility of multiple target molecules inhabiting a single droplet. Using Poisson's law of small numbers, the distribution of target molecule within the sample can be accurately approximated allowing for a quantification of the target strand in the PCR product. This model simply predicts that as the number of samples containing at least one target molecule increases, the probability of the samples containing more than one target molecule increases. In conventional PCR, the number of PCR amplification cycles is proportional to the starting copy number. Digital PCR uses statistical power to provide relative quantification.

In some exemplary embodiments, the methods described herein are performed on DNA, e.g. genomic DNA, with sizes ranging from tens to hundreds of thousands of base pairs. In other embodiments, smaller strands of DNA are analyzed, for example, under 300 bp in length, or under 250 bp or under 200 bp in length.

Patient samples can be extracted with a variety of methods known in the art to provide nucleic acid (e.g. genomic DNA) for use in the methods described herein. For example, a DNA sample may be extracted from blood, tissue (e.g. tumor sample), saliva, urine, semen, etc.

As used herein, the term “reference DNA sample” or “control DNA sample” refers to genomic DNA obtained from a healthy individual who does not have a length polymorphism or a disease or disorder associated with a length polymorphism, also referred to herein as “wild type”. The term “wild type” as used herein refers to the normal, or non-mutated, or functional form of a gene.

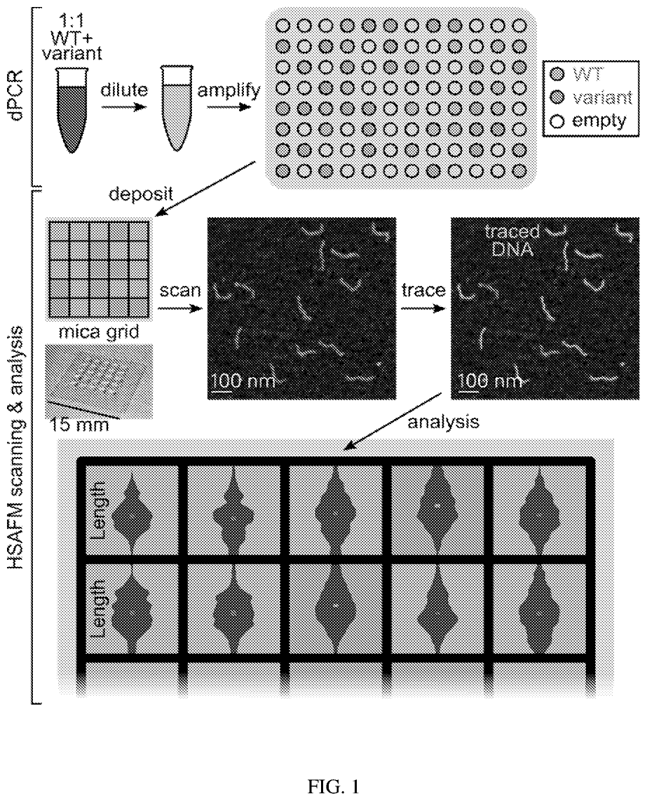

With reference to FIG. 1, in an exemplary embodiment combining dPCR with AFM, a heterogeneous sample containing both wild type (WT) and variant DNA is first diluted to yield a target of 1 or 0 template strands in each well of a PCR plate. After amplification using a digital PCR kit, either no DNA or a nominally homogeneous population of amplicons is obtained within each well. The homogeneous yield in each well enables quantification of the variant allele frequency (VAF) from bulk solution, as variant wells can be clearly distinguished from WT, and the percentage of variant v. WT wells can be simply calculated. Following dPCR, each well may be purified and its contents deposited onto an atomically flat surface, such as mica, for AFM scanning.

AFM is a very-high-resolution type of scanning probe microscopy (SPM), with demonstrated resolution on the order of fractions of a nanometer, more than 1000 times better than the optical diffraction-limit. AFM systems compatible with the present disclosure are known in the art, e.g. as disclosed in U.S. Pat. No. 9,926,589 incorporated herein by reference. In some embodiments, the AFM is high-speed AFM. High-speed AFM (HS-AFM) is a type of atomic force microscopy (AFM) that, unlike conventional AFM, can take an image very quickly and with relatively low imaging force. High speed AFM may be defined as >200,000 pixels per second. Typical pixel sizes range from 1 ×1 nm to 10×10 nm. High speed AFM allows for the characterization of the conformation of a plurality of fixed molecules across a wide range of area. In some embodiments, the sample is dry not immersed in liquid. In some embodiments, a scan speed of 0.5-2 Hz is used, e.g. a 1 Hz scan speed.

Tracing software may be used to process the images and to determine the amplicon length distribution in each well. For example, the computer-implemented method may include steps of flattening an AFM image; thresholding to identify DNA strands; skeletonizing the thresholded strands; determining the longest backbone of each skeleton; measuring the length of the longest backbone; and applying a quality filter. A decision test, e.g. using a Bayesian statistical analysis, may then be applied to the distributions to determine whether the amplicons in each well most likely originated from a WT or variant template.

Bayesian statistics is based on the Bayesian interpretation of probability where probability expresses a degree of belief in an event. The degree of belief may be based on prior knowledge about the event, such as the results of previous experiments, or on personal beliefs about the event. Bayesian statistical methods use Bayes' theorem to compute and update probabilities after obtaining new data. Bayes' theorem describes the conditional probability of an event based on data as well as prior information or beliefs about the event or conditions related to the event. For example, in Bayesian inference, Bayes' theorem can be used to estimate the parameters of a probability distribution or statistical model. Since Bayesian statistics treats probability as a degree of belief, Bayes' theorem can directly assign a probability distribution that quantifies the belief to the parameter or set of parameters.

A Bayesian approach may be used to determine the most credible mean amplicon length within a given reaction/well and to characterize the distribution as WT or variant. In some embodiments, 95% highest density interval (HDI) of each distribution's mean length (a measure similar to a confidence interval that gives the 95% most credible values of the mean) is determined. Then, the well may be characterized as WT or variant based on whether its 95% HDI fell within a region of practical equivalence (ROPE): a range of lengths centered at the most credible WT length (null value). In some embodiments, a receiver operating characteristic (ROC) curve is used to set the optimal ROPE. The VAF for a given sample may be equal to nᵥₐᵣ/(nᵥₐᵣ+nwT), where nᵥₐᵣ is the number of variant wells and nwT is the number of WT wells according to the decision rule.

In some embodiments, the target region is labeled with CRISPR associated protein 9 (Cas9). In some embodiments other molecules or nanoparticles are used as labels instead of Cas9. The chemistry to attach such labels is known in the art. Labeling a target motif with programmable Cas9 and imaging to reveal the Cas9 molecules bound to strands allows on-target strands to be distinguished from off-target, improving specificity. Some embodiments provide methods of Cas9 labeling as disclosed in U.S. Pat. No. 9,926,589 incorporated herein by reference. In some embodiments, the target region is not labeled with Cas9 or any other labeling molecule.

The methods described herein can be used to detect length polymorphisms including additions and deletions such as nucleotide repeat expansions, copy number variations, and tandem duplications, such as internal tandem duplications. The present methods can be applied to any length polymorphism as long as the site of the polymorphism is known and can be amplified.

Length polymorphisms are associated with several diseases and disorders such as Fragile X syndrome, Huntington disease, hereditary ataxias, and various cancers such as lung, breast, blood, and prostate cancers. Accordingly, some embodiments of the disclosure provide methods of diagnosing a subject with a disease or disorder associated with a length polymorphism using a detection method as described herein. Further embodiments provide a method of treating the disease or disorder comprising diagnosing the disease or disorder using a detection method as described herein and subsequently treating the subject with a suitable therapy for the disease or disorder, e.g. an anti-cancer agent.

The terms “subject” and “patient” are used interchangeably herein, and refer to an animal such as a mammal, which is afflicted with or suspected of having, at risk of, or being pre-disposed to a disease or disorder associated with a length polymorphism. In general, the terms refer to a human. The terms also include domestic animals bred for food, sport, or as pets, including horses, cows, sheep, poultry, fish, pigs, cats, dogs, and zoo animals, goats, apes (e.g. gorilla or chimpanzee), and rodents such as rats and mice. Typical subjects include persons susceptible to, suffering from or that have suffered a disease or disorder associated with a length polymorphism.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLE

### Summary

Length polymorphisms are found in a host of serious diseases, and assessment of mutation length and variant allele frequency (VAF) is often critical for accurate diagnosis. However, characterization of length polymorphisms remains challenging due to their frequently variable or repetitive nature. Here, we present digital polymerase chain reaction (dPCR) followed by high-speed atomic force microscopy (HSAFM) as an effective method for quantifying length polymorphisms. We focused on internal tandem duplications (ITDs) located within the FLT3 gene, which are associated with acute myeloid leukemia and often indicative of a poor prognosis. In analysis of over 1.5 million HSAFM-imaged amplicons from cell line and clinical samples containing FLT3-ITDs, dPCR-HSAFM returned the expected variant length and VAF, down to 5% VAF samples. As a flexible method with single-molecule resolution, dPCR-HSAFM thus represents a new frontier for HSAFM imaging and a powerful tool for the diagnosis of length polymorphisms.

Materials and Methods

To test and validate dPCR-HSAFM, we focused on the case of internal tandem duplications (ITDs) within exons 14 and 15 of the FLT3 gene, a length polymorphism associated with acute myeloid leukemia (AML).^{6,8} Using a collection of over 200,000 atomic force microscopy images, we show dPCR-HSAFM to be an accurate, straightforward, and low-cost option to characterize FLT3-ITDs.

Samples

Human female genomic DNA was obtained from Promega (Madison, Wis.), synthetic DNA was ordered from Integrated DNA Technologies (Coralville, Iowa), and genomic DNA from cell lines MV4-11 and PL-21 were obtained from DSMZ (Braunschweig, Germany). Since PL-21 is a heterozygote containing one WT FLT3 allele and one FLT3 allele with a 126 bp insert, we used a 126 bp synthetic template to obtain a 100% VAF sample for our 126 bp insert series. With IRB approval, de-identified clinical samples in the form of slides with bone marrow aspirate smears from confirmed AML patients were obtained from the Department of Pathology at Virginia Commonwealth University. FLT3-ITD status and insertion length measured via the Leukostrat CDx FLT3 mutation assay were collected from uploaded reports in the patients' electronic medical records (Table 1). We extracted and purified genomic DNA from slides using MagAttract HMW DNA kit (Qiagen, Gaithersburg, Md.), and concentrations of DNA were determined with Qubit dsDNA HS assay (Invitrogen, ThermoFisher Scientific, Carlsbad, Calif.). Samples are listed in Table 1.

Digital PCR

We employed dPCR to amplify our target gene (FLT3) and determine the proportion of DNA within a sample containing a variant allele. We used 1× Luna Universal Probe qPCR master mix (New England Biolab, Ipswich, Mass.), 0.025 units/μL Antarctic Thermolabile UDG (New England Biolab, Ipswich, Mass.), 25 nM of forward and reverse primers, 25 nM of fluorogenic probe, and template DNA at a concentration of 1 copy per 10-15 μl of dPCR solution, for a final volume of 5 μl per well. PCR was performed on a C1000 Touch Thermo Cycler with CFX96 Real-Time System optical head (Bio-Rad Laboratories, Hercules, Calif.). We targeted exons 14 and 15 of the FLT3 gene for amplification using the following forward and reverse primer sequences, respectively: ACTGCCTATTCCTAACTGACTCATC (SEQ ID NO: 1) and CTTTCAGCATTTTGACGGCAACC (SEQ ID NO: 2). We used the fluorogenic probe 6-FAM-CAGGGAAGG/ZEN/TACTAGGATCAGGTGCT-IABkFQ (SEQ ID NO: 3), where “6-FAM” indicates fluorescein, “ZEN” indicates ZEN quencher, and “IABkFQ” indicates Iowa Black Fluorescence quencher. Our amplification protocol involved 25° C. for 10 min, 95° C. for 1 min, 50 cycles at 95° C. for 15 sec each, and 60° C. for 2 min. To determine the presence of amplicons in dPCR wells, qPCR kinetic curves were recorded for each well and visually inspected; only wells with distinct S-like amplification curves were interpreted to contain DNA and thereby used for further analysis. Non-template control (NTC) plates were run regularly to detect for environmental contamination, and no kinetic curves were detected in NTC plates. The amplicons from the positive wells were purified with 5 μL of AMPure XP (Beckman Coulter, Brea, Calif.) per well and eluted in 10 μl of TE buffer.

Deposition for HSAFM Scanning

Since atomic force microscopy depends on resolving the topography of a desired feature against the substrate background, imaging the 2 nm-diameter backbone of DNA requires an atomically flat substrate. We thus deposited our dPCR samples onto 15 mm×15 mm squares of cleaved mica (Ted Pella) for scanning. We subdivided our mica into 25 sample regions by printing UV-cured epoxy into a 5×5 grid on the mica surface. The applied dPCR-HSAFM procedure was equivalent for all samples except sW-0; in that case, aliquots were deposited directly onto mica without first undergoing dPCR, thereby providing a control to determine the effect of dPCR on the length distributions. To deposit each sample other than sW-0, we followed a slightly modified version of our previously described procedure.^{23,24} We first added MgCl₂ to the purified DNA in order to facilitate adhesion of negatively-charged DNA to negatively-charged mica and diluted the sample to yield the optimal density of DNA molecules; we found the ideal deposition concentration to be 0.1 ng/μL of DNA with 2.5 mM MgCl₂. We then deposited approximately 0.2 μL into each grid square, waited 1 minute after the final sample deposition to allow for DNA relaxation on the surface, and washed the entire grid 3 times with 600 μL of Millipore water. We then immediately loaded the sample into a fixed apparatus with a wide-angle nozzle (ImpactRM) to keep the angle and pressure (2.5 psi) of airflow consistent, and applied compressed air to dry the grid. We transferred the grid to an infrared oven and baked at 120° C. for 10 minutes. DNA was not deposited in at least one grid square of each mica sheet in order to provide a baseline for background noise and to detect for carryover of DNA between wells during washing. Empty wells consistently revealed negligible carryover of DNA (<1 strand per 20 frames) due to the unfavorable conditions for DNA deposition in a wash of excess pure water.

HSAFM

Individual DNA molecules were imaged using HSAFM and measured using custom computer vision software (MATLAB). Our HSAFM is a contact-mode system with a laser vibrometer to detect height and a flexure stage for high-speed lateral displacement.²⁵ For DNA imaging, we found optimal conditions to be a 1 Hz scan speed, a 1800 nm×1800 nm scan size, and 1000×1000 pixel resolution. These settings yielded ˜5 nm (15 bp) length accuracy,²⁴ while the laser vibrometer yielded 0.015 nm height resolution. We used measurements from our synthetic control sample (sW-0) as a fiducial marker²⁶ to calibrate the lateral dimensions of our HSAFM images and to correct for strand length variation due to tip wear²⁷. In each grid square containing the amplicons from a single dPCR well, we nominally captured 55 frames, which required approximately 1 minute to land the HSAFM probe onto the surface and 2 minutes to scan. We measured strand length in HSAFM images using custom computer vision software. Briefly, our algorithm flattened the raw AFM image, thresholded to identify DNA strands, skeletonized those thresholded strands, discerned the longest backbone of each skeleton, measured the length of the backbone, and applied a quality filter. To mitigate the inclusion of nanoscale surface contamination in our DNA length measurements, we discarded strands less than 70 nm (≈210 bp) in length. We also discarded wells with fewer than 150 strands after tracing and filtering as a conservative cutoff in order to mitigate the influence of background contamination and achieve sufficient confidence in our estimates of mean length.

Bayesian Analysis

We used a Bayesian approach to determine the most credible mean amplicon length within a given well and to characterize the distribution as WT or variant. In contrast to null hypothesis testing using a frequentist approach, Bayesian inference does not depend on sampling or testing intentions; instead, prior knowledge is explicitly described and included in the calculation of the posterior distribution of parameter values by Bayes' rule.²⁸ This posterior distribution, which we derived by Markov chain Monte Carlo (MCMC) sampling, directly imparts the credibility of each parameter value.

Results

To validate the dPCR-HSAFM technique, we tested (a) synthetic and cell line samples of known length and spiked-in VAF, and (b) clinical samples previously analyzed using the standard PCR-CE clinical test, Leukostrat, which reports insert length but not VAF. Table 1 shows a list of all tested samples.

TABLE 1
Samples tested by dPCR-HSAFM. WT FLT3 amplicons are 368 bp.
Insert lengths of the cell line and synthetic samples were
reported by the providers, while insert lengths of the clinical
samples were reported by the Leukostrat assay. The CE used in the
Leukostrat assay reports a <1 bp sizing reproducibility between
runs;³² we conservatively state ±2 bp here to account
for calibration considerations.

| | | | | Insertion |
|---|---|---|---|---|
| Sample name | VAF | Parent sample | Sample type | length (bp) |
| W-0 | 0 | WT | Cell line | 0 |
| sW-0 | 0 | Syn-WT | Synthetic | 0 |
| M-100 | 1 | MV4-11 | Cell line | 30 |
| M-50 | 0.5 | | | |
| M-25 | 0.25 | | | |
| M-05 | 0.05 | | | |
| sP-100 | 1 | Syn-126 | Synthetic | 126 |
| P-50 | 0.5 | PL-21* | Cell line | 126 |
| P-25 | 0.25 | | | |
| P-10 | 0.1 | | | |
| P-05 | 0.05 | | | |
| clinA | — | clinA | Clinical | 27 ± 2 |
| clinB | — | clinB | | 48 ± 2 |
| clinC | — | clinC | | 24 ± 2 |
| clinD | — | clinD | | 18, 54, 57 ± 2 |
| clinE | — | clinE | | 0 |
| clinF | — | clinF | | 0 |

*PL-21 is a FLT3 heterozygote, with one WT allele and one allele with a 126 bp insert.

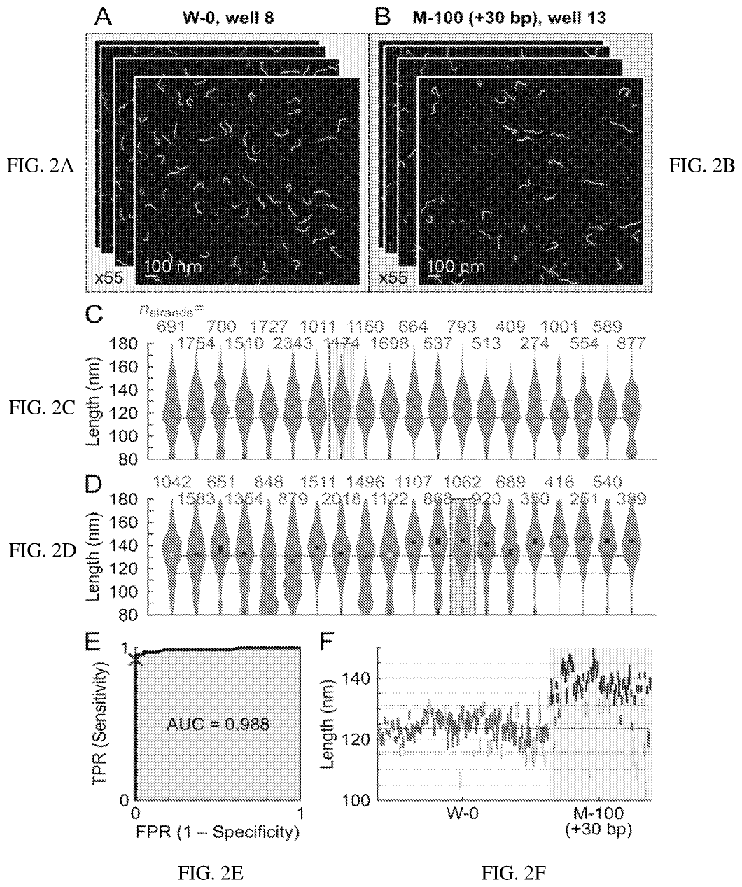

Example scan images from two samples (W-0 and M-1 are shown in FIG. 2A-B and length distributions of traced DNA molecules from W-0 and M-100 wells are shown in FIG. 2C-D. For each well, at least 55 images with a size of 1.8 μm×1.8 μm and resolution of 1000×1000 pixels were scanned and traced. 2,213 wells were scanned in total for this study, yielding over 200,000 images (>100 gigapixels) and 1.5 million traced strands.

To determine if the amplicons within a given well likely originated from a WT or variant template, we used a decision rule based on Bayesian inference that is similar to an equivalence test.³⁰ First, we determined the 95% highest density interval (HDI) of each distribution's mean length—a measure similar to a confidence interval that gives the 95% most credible values of the mean. Then, the well was characterized as WT or variant based on whether its 95% HDI fell within a region of practical equivalence (ROPE): a range of lengths centered at the most credible WT length (null value). FIGS. 2C, D, and F show the application of the decision rule to W-0 and M-100 wells, where the color of each 95% HDI reflects the outcome of the rule. The VAF for a given sample was equal to nᵥₐᵣ/(nᵥₐᵣ+nwT), where nᵥₐᵣ is the number of variant wells and n_{WT} is the number of WT wells according to the decision rule. Since the ROPE boundaries were crucial to the outcome of the decision test, we used a receiver operating characteristic (ROC) curve to set the optimal ROPE (FIG. 2E).³³ W-0 and M-100 (+30 bp insertion) were employed as control and test samples, respectively, since we desired our ROC-derived test to be optimized to detect the smallest possible insertion, and MV4-11 presented the smallest insertion in our sample pool. We found the optimal ROPE (FIG. 2E, “x”; and FIG. 2C,D,F, dotted lines) to be 115.8-131.0 nm by maximizing specificity, sensitivity, and the number of non-excluded samples in our ROC analysis.

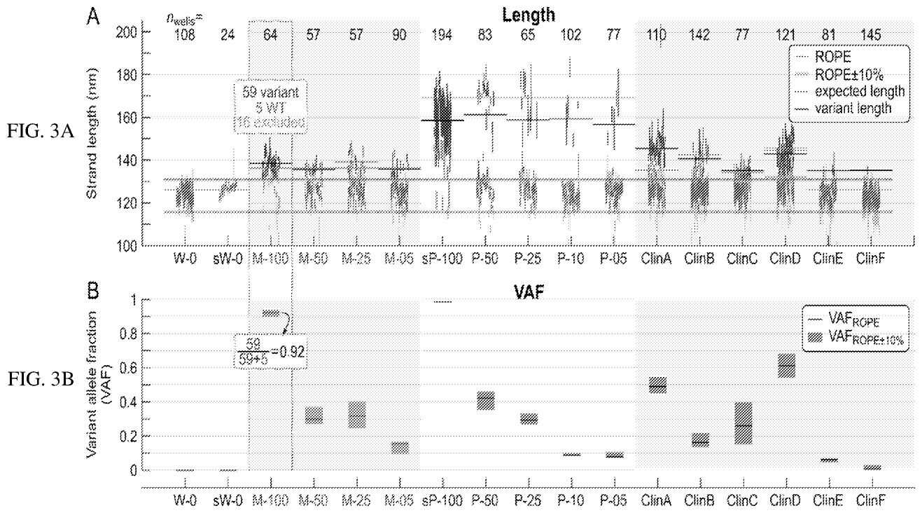

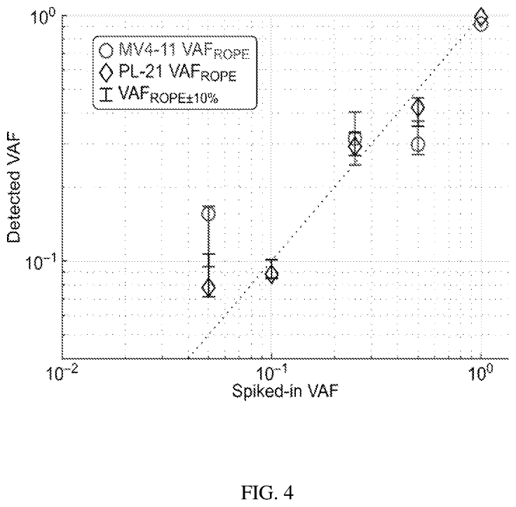

After establishing the optimal ROPE with W-0 and M-100, we applied our decision rule to all samples and determined their VAFs (FIG. 3). We found good agreement between expected and detected length (FIG. 3A), although for samples with 126 bp insertions, the overall averages of variant 95% HDIs were lower than expected. The 95% HDIs for the clinical samples generally matched their expected lengths as reported by the gel-based Leukostrat test, although our length measurement for ClinA was substantially longer than the Leukostrat result. Additionally, ClinE and ClinF were found to be negative for FLT3-ITDs by the Leukostrat assay, but dPCR-HSAFM registered a small number of wells in each of those samples as variant (5 wells in ClinE and 2 wells in ClinF), with the 95% HDIs of those variant wells falling barely above the ROPE. FIG. 3B and FIG. 4 show the VAF for each sample based on the results of the decision test, and an example calculation of VAF is shown for sample M-100. To gauge the effect of ROPE choice on VAF outcome, we conducted the decision test with alternative ROPEs centered at the null value, but ranging in width from 90% to 110% of the width of the ROC-derived ROPE (FIG. 3A). The resulting VAF spreads are shown in FIG. 3B (shaded areas) and FIG. 4 (vertical lines), where a larger spread indicates that several 95% HDIs are near a ROPE boundary. Accuracy and reproducibility of dPCR-HSAFM were evaluated by calculating the mean size offset and coefficient of variation (CV), respectively, of our non-mixed samples (Table 2).

TABLE 2
Mean size offset and CV of tested homogeneous samples.
95% confidence interval (CI) of the mean size offset gives
the result of bootstrapping with 10,000 resamples.³⁴

| | | Mean size offset (%) | Well | |
|---|---|---|---|---|
| Sample | Length (bp) | [95% CI mean size offset] | count | CV (%) |
| W-0 | 368 | −2.3 [−2.8, −1.7] | 108 | 3.5 |
| sW-0 | 368 | 0.5 [−0.1, 1.2] | 24 | 1.7 |
| M-100 | 398 | −1.1 [−2.5, 0.2] | 64 | 6.2 |
| sP-100 | 494 | −6.8 [−7.7, −5.9] | 194 | 6.8 |

### DISCUSSION

Accuracy and Reproducibility

Our testing of FLT3-ITD samples shows that dPCR-HSAFM successfully detected insertions of 30 bp and 126 bp at clinically relevant VAFs. As shown in FIG. 4, our HSAFM measurements and decision test yielded good agreement between spiked-in and detected VAF down to a spiked-in VAF of 5%, which represents the maximum sensitivity of the current clinical standard, the Leukostrat CDx FLT3 mutation assay.¹³ The Leukostrat test accurately reports the length and presence of an ITD for 24 bp inserts at VAFs down to 5%, but the detection sensitivity drops to 10% VAF for 66 bp insertions and only 28% VAF for 217 bp insertions.¹³ This rapid decline in sensitivity with insertion length implies that longer FLT3-ITDs may be widely underdiagnosed by PCR-CE methods. Additionally, the Leukostrat test is unable to quantify the VAF due to the limitations of CE. VAF is an important indicator of patient prognosis,^{6,9,14,15} and there is an expanding catalog of treatment options for FLT3-ITD positive patients.^{35,36} Thus, a platform like dPCR-HSAFM that can rapidly and cost-effectively report FLT3-ITD length and VAF across a wide range of lengths is attractive.

Accuracy and reproducibility of dPCR-HSAFM length measurements (FIG. 3A, Table 2) are similar to those of wide-range CE systems like the Agilent Bioanalyzer, which advertises a sizing accuracy of ±10% CV and reproducibility of 5% CV,³⁷ but dPCR-HSAFM performance characteristics fall short of the ≈1 bp accuracy and reproducibility of narrow-range CEs like the Applied Biosystems 3500 Genetic Analyzer that is specified for use with the Leukostrat assay.^{13,32} sW-0, the only sample that was scanned without undergoing dPCR, displayed the lowest CV and variation in mean size offset, which was expected due to the variability introduced by amplification. It is notable that the CV of M-100 and both the CV and size offset of sP-100 are >6%. For M-100, the relatively large CV and spread of the mean size offset are due to several length distributions that fell within or below the ROPE (FIG. 3A). Because M-100 was derived from a cell line, these distributions of short-reading amplicons could be the result of native sample heterogeneity and not the inherent performance characteristics of the technique, and thus should be interpreted with caution. On the other hand, sP-100 originated from a synthetic DNA template, so its large CV and mean size offset must have another explanation. In sP-100 and in the PL-21 samples that also contained a 126 bp insertion, the detected length is substantially lower relative to expected, and the 95% HDIs are more widely spread than in other samples (FIG. 3A). In our previous work, we did not observe a systematic underestimation of length as the length of DNA was increased.²⁴ While further exploration of HSAFM scanning and automated tracing over a range of non-amplified DNA lengths will further illuminate the nature of the discrepancy, we believe the effect is largely due to (a) incomplete amplification, which was previously reported for longer dPCR amplicons;³⁸ and (b) unelongated DNA due to sample preparation and deposition, which is anticipated to scale with greater length. The observed incomplete elongation was inconsistent between wells, indicating that it likely originated from unpredictable drying of water droplets containing DNA as they were blown across the raised edges of the grid during the compressed air stage of deposition. Attempts were made to filter out unelongated strands according to their height profiles in post-processing, but the filtering was not universally effective.

dPCR-HSAFM analysis of clinical samples generally matched the Leukostrat assay results, although notable exceptions reveal the limits and promise of the approach. First, the mean variant length of ClinA as determined by dPCR-HSAFM is strikingly greater than the value returned by Leukostrat (FIG. 3A). We suggest that these disparate lengths are due to tumor mosaicism, i.e. different mutations present in the cell populations analyzed by the two techniques. The Leukostrat assay results were retrospective and thus cannot be sequenced, but we retain access to amplicons in every dPCR well, each ideally sourced from a single template molecule, and thus could sequence wells that yielded variant length distributions if the mosaicism of a particular sample was to be thoroughly characterized. A bulk PCR or sequencing technique cannot resolve the variant constituents with single-molecule resolution.³⁹

As for the VAF characterization of ClinA, both tests designated the sample as clearly positive for FLT3-ITD, and there was good agreement in both variant length and FLT3-ITD positive result for ClinB, ClinC, and ClinD as well. In contrast, ClinE and ClinF were both found to be FLT3-ITD negative by the Leukostrat assay, but the dPCR-HSAFM length distributions of a small number of wells from those samples were found to fall above the ROPE, thus returning VAF>0. However, the few variant-identified wells returned 95% HDIs that were only marginally greater than the upper ROPE bound—a factor that can and should be considered when assessing the credibility of a positive or negative FLT3-ITD assignation. Additional rigorous testing with a greater number of positive and negative controls will allow for the refinement of the ROPE values and methodology of the dPCR-HSAFM decision test, but it is notable that the resolution and quantitative nature of the dPCR-HSAFM data enable a more informative and transparent FLT3-ITD result to be reported than a binary positive or negative.

As a proof of principle, the close agreement between our spiked-in VAF and detected VAF (FIG. 4) suggests that dPCR-HSAFM can accurately predict VAF. These results promise a significant advantage over CE: quantification of fluorescence curves is not reported in Leukostrat assays, quantification accuracy and reproducibility is not listed for the narrow-range CE employed in the Leukostrat,³² and 20% CV for quantification accuracy and 10-15% CV for quantification reproducibility is expected in the most sensitive wide-range CE systems.37

dPCR

dPCR offers distinct advantages over bulk PCR in the quantification of variants within a mixed sample.⁴⁰ By “digitizing” the sample constituents into a homogeneous population of amplicons within each well, length analysis and determination of VAF is straightforward. dPCR also mitigates PCR bias: by separating single target DNA molecules into their own wells for amplification, the preferential amplification of shorter over longer strands within a single solution is avoided. As indicated by our negative non-template control results, we showed that dPCR contamination can be avoided using amplification with dUTP and careful procedures. Finally, dPCR is a single-molecule technique, as only a single template molecule is required to initiate amplification within a well. While this study focused on FLT3-ITDs, dPCR-HSAFM could be applied to any length polymorphism as long as the site of the polymorphism is known and can be amplified.^{1,2}

For the qPCR kit we employed in this study, a maximum amplicon length of only 200 bp is recommended.⁴¹ While longer amplification is possible,⁴² a decrease in amplification efficiency relative to the shorter WT template is expected in proportion to variant length. It is likely that this decrease in efficiency will be consistent and correctable by a spiked-in VAF v. detected VAF calibration curve,¹⁵ but any decrease in efficiency demands an increase in well sampling in order to achieve an equivalent VAF sensitivity—and indeed, the inverse relationship between well number and VAF sensitivity is another dPCR consideration. With our current setup and assuming a binomial distribution of variants within dPCR wells, 3000 samples would be needed to detect a 10⁻³ VAF with 95% confidence; in comparison, a recent study showed that NGS can detect a VAF as low as 10^{-5,15} Gains in miniaturization and software automation are fully achievable.^{40,43} Furthermore, while minimal residual detection of disease down to 10-5 VAF may be useful for some prognoses, a lower-cost option that gives an accurate report of VAF and variant length at higher VAFs would still be useful. For example, in the case of FLT3-ITDs, the risk of poor outcome appears stratified over a range of VAFs, from 10-⁵ to 50%.6,9-14,15

Recently, microfluidics has enabled the development of miniaturized dPCR platforms like droplet digital PCR (ddPCR), wherein a diluted dPCR solution is hypercompartmentalized into oil-separated droplets that are each amplified, yielding thousands or even millions of distinct fluorescence readings.⁴⁰ Using a nonspecific DNA-binding dye, VAF of a mixed sample was determined down to 20%.³⁸ These encouraging results are nevertheless, like CE, limited by the bulk nature of the fluorescence output for a single ddPCR well, which indicates both amplicon length and VAF and must be calibrated for amplification efficiency, droplet volume, and the DNA template volume. Multiplexed ddPCR with multi-channel fluorescent probes or dyes may help mitigate the confounding of amplicon length and VAF,⁴⁴ but reproducible and accurate partitioning of the multiplexed signals presents its own challenges.⁴⁵ The gains in throughput and sensitivity of dPCR due to automation and miniaturization mirror advancements in AFM technology, and we believe there is great use in both techniques for broader applications.

HSAFM

This study represents the first statistically robust diagnosis of a genetic variant with atomic force microscopy (AFM). AFM was invented in the 1980s⁴⁶ and has served as a highly flexible research tool for discerning the topography and mechanical properties of a surface with sub-nanometer resolution,⁴⁷ but limitations in scan speed (typically 0.001 Hz for a 1000×1000 pixel image) has hampered its widespread adaptation in large-scale analyses. Previous AFM studies of DNA involved tracing dozens or hundreds of DNA molecules by hand, sometimes with the aid of smoothing algorithms to better fit the traces to strand curvature.^{23,24,48-53} The development of HSAFM-which can image 1000×1000 pixel images at speeds of 1 Hz or greater-enabled an exciting range of new possibilities, but HSAFM biological research has mainly focused on characterizing biomolecular kinetics or whole-cell imaging at nanoscale resolution and with different imaging modes.^{47,54,55} Recently, some groups have explored a large-area approach to collecting robust statistical measurements of features,^{43,56,57} but to our knowledge, fully automated flattening and tracing as exhibited in this work has never been developed to the extent that it could be applied to such a large sample size (>200,000 images and >1.5 million DNA molecules traced). The scanning procedures and automated processing routines developed here enabled gigapixel-scale analysis.

HSAFM offers unique benefits in the characterization of genetic variants. First, the single-molecule sensitivity of HSAFM, which complements the single-molecule amplification of dPCR, has many advantages. A single-molecule imaging approach requires less material: to diagnose a well containing WT or variant amplicons, we scanned an area <200 μm², which usually contained a few hundred strands. These numbers point to the potential of the process to be scaled down and paired with other microscale single-molecule techniques, such as ddPCR or amplification-free analysis. It is notable that either ddPCR or dPCR detect the presence of single molecules before amplification, but HSAFM scans produce data with single-molecule resolution after amplification, enabling more powerful statistical analysis than the bulk fluorescence intensity measurements achieved with CE or ddPCR. For example, individual species can be identified and quantified after multiplexing with low-cycle number PCR,²³ and in the detection of nuanced differences between populations, the Bayesian approach we applied here can be extended to sophisticated hierarchical models.²⁸

The direct-imaging approach of HSAFM confers additional advantages. Unlike a narrow-range CE like that employed in the Leukostrat assay, HSAFM is an extremely wide-range length measurement system: by stitching together multiple overlapping HSAFM images, strands of virtually any length can be imaged and traced.^{24,58} HSAFM images can also convey more than length measurement: as we previously showed,²⁴ labeling a target motif with programmable Cas9 and imaging to reveal the Cas9 molecules bound to strands allows on-target strands to be distinguished from off-target, improving specificity.

### CONCLUSIONS

In mixed cell line and clinical samples containing FLT3-ITDs, dPCR-HSAFM successfully reported variant length and allele fraction. Using high-throughput scanning and automated tracing of DNA strands, we acquired a large data set that facilitated robust analysis by Bayesian inference. The scale and single-molecule resolution of the data, plus the ability of the method to report VAF, make it an attractive alternative to the standard FLT3-ITD clinical assay, which returns bulk fluorescence data and cannot quantify the VAF with confidence. Accuracy and reproducibility of dPCR-HSAFM results were similar to that of wide-range capillary gel electrophoresis. With single-molecule sensitivity before and after amplification, the ability to image molecules of virtually any length, high throughput, and low cost, dPCR-HSAFM serves as a valuable tool for the diagnosis of FLT3-ITDs and other length polymorphisms.

### REFERENCES

1. Wallace, S. E. & Bird, T. D. Molecular genetic testing for hereditary ataxia. Neurology: Clinical Practice 8, 27-32 (2018).
2. Pilotto, F. & Saxena, S. Epidemiology of inherited cerebellar ataxias and challenges in clinical research. Clinical and Translational Neuroscience 2, 2514183X1878525 (2018).
3. Qiu, Z.-W., Bi, J.-H., Gazdar, A. F. & Song, K. Genome-wide copy number variation pattern analysis and a classification signature for non-small cell lung cancer. Genes, Chromosomes and Cancer 56, 559-569 (2017).
4. Lu, Y. et al. Role of CYP2E1 polymorphisms in breast cancer: a systematic review and meta-analysis. Cancer Cell International 17, (2017).
5. Kumaran, M. et al. Germline copy number variations are associated with breast cancer risk and prognosis. Scientific Reports 7, (2017).
6. Döhner, H. et al. Diagnosis and management of AML in adults: 2017 ELN recommendations from an international expert panel. Blood 129, 424-447 (2016).
7. Weng, H. et al. Androgen receptor gene polymorphisms and risk of prostate cancer: a meta-analysis. Scientific Reports 7, (2017).
8. Lagunas-Rangel, F. A. & Chavez-Valencia, V. FLT3-ITD and its current role in acute myeloid leukaemia. Medical Oncology 34, (2017).
9. Chen, F. et al. Impact of FLT3-ITD allele ratio and ITD length on therapeutic outcome in cytogenetically normal AML patients without NPM1 mutation. Bone Marrow Transplantation 55, 740-748 (2019).
10. Gale, R. E. et al. The impact of FLT3 internal tandem duplication mutant level, number, size, and interaction with NPM1 mutations in a large cohort of young adult patients with acute myeloid leukemia. Blood 111, 2776-2784 (2008).
11. Schlenk, R. F. et al. Differential impact of allelic ratio and insertion site in FLT3-ITD-positive AML with respect to allogeneic transplantation. Blood 124, 3441-3449 (2014).
12. Grafone, T., Palmisano, M., Nicci, C. & Storti, S. An overview on the role of FLT3-tyrosine kinase receptor in acute myeloid leukemia: biology and treatment. Oncology Reviews 6, 8 (2012).
13. LeukoStrat® CDx FLT3 Mutation Assay. (Invivoscribe: 2017).
14. Lyu, M. et al. The prognosis predictive value of FMS-like tyrosine kinase 3-internal tandem duplications mutant allelic ratio (FLT3-ITD MR) in patients with acute myeloid leukemia detected by GeneScan. Gene 726, 144195 (2020).
15. Levis, M. J. et al. A next-generation sequencing-based assay for minimal residual disease assessment in AML patients with FLT3-ITD mutations. Blood Advances 2, 825-831 (2018).
16. Fischer, M. et al. Impact of FLT3-ITD diversity on response to induction chemotherapy in patients with acute myeloid leukemia. Haematologica 102, e129-e131 (2016).
17. Grimwade, D. & Freeman, S. D. Defining minimal residual disease in acute myeloid leukemia: which platforms are ready for “prime time” ? Hematology 2014, 222-233 (2014).
18. Kayser, S. et al. Insertion of FLT3 internal tandem duplication in the tyrosine kinase domain-1 is associated with resistance to chemotherapy and inferior outcome. Blood 114, 2386-2392 (2009).
19. Bianchini, M. et al. Rapid Detection of Flt3 Mutations in Acute Myeloid Leukemia Patients by Denaturing HPLC. Clinical Chemistry 49, 1642-1650 (2003).
20. Levine, R. L. & Valk, P. J. M. Next-generation sequencing in the diagnosis and minimal residual disease assessment of acute myeloid leukemia. Haematologica 104, 868-871 (2019).
21. Sedlazeck, F. J., Lee, H., Darby, C. A. & Schatz, M. C. Piercing the dark matter: bioinformatics of long-range sequencing and mapping. Nat. Rev. Genet. 19, 329-346 (2018).
22. Sykes, P. et al. Quantitation of targets for PCR by use of limiting dilution. Biotechniques 13, 444-449 (1992).
23. Mikheikin, A. et al. Atomic Force Microscopic Detection Enabling Multiplexed Low-Cycle-Number Quantitative Polymerase Chain Reaction for Biomarker Assays. Analytical Chemistry 86, 6180-6183 (2014).
24. Mikheikin, A. et al. DNA nanomapping using CRISPR-Cas9 as a programmable nanoparticle. Nat. Commun. 8, 1665 (2017).
25. Picco, L. M. et al. Breaking the speed limit with atomic force microscopy.

Nanotechnology 18, 044030 (2007).
26. Fuentes-Perez, M. E., Gwynn, E. J., Dillingham, M. S. & Moreno-Herrero, F. Using DNA as a Fiducial Marker To Study SMC Complex Interactions with the Atomic Force Microscope. Biophysical Journal 102, 839-848 (2012).
27. Santos, S., Barcons, V., Christenson, H. K., Font, J. & Thomson, N. H. The intrinsic resolution limit in the atomic force microscope: implications for heights of nano-scale features. PLoS One 6, e23821 (2011).
28. Kruschke, J. Doing Bayesian data analysis: A tutorial with R, JAGS, and Stan. (Academic Press: 2015).
29. Kruschke, J. K. Bayesian estimation supersedes the t test. Journal of Experimental Psychology: General 142, 573-603 (2013).
30. Kruschke, J. K. Rejecting or Accepting Parameter Values in Bayesian Estimation. Advances in Methods and Practices in Psychological Science 1, 270-280 (2018).
31. Gelman, A., Hwang, J. & Vehtari, A. Understanding predictive information criteria for Bayesian models. Statistics and Computing 24, 997-1016 (2013).
32. 3500 and 3500xL Series Genetic Analyzers. (Applied Biosystems:).
33. Fawcett, T. An introduction to ROC analysis. Pattern Recognition Letters 27, 861-874 (2006).
34. Motulsky, H. Intuitive biostatistics: a nonmathematical guide to statistical thinking. (Oxford University Press, USA: 2013).
35. Levis, M. & Perl, A. E. Gilteritinib: potent targeting of FLT3 mutations in AML. Blood Advances 4, 1178-1191 (2020).
36. Gurnari, C., Voso, M. T., Maciejewski, J. P. & Visconte, V. From Bench to Bedside and Beyond: Therapeutic Scenario in Acute Myeloid Leukemia. Cancers 12, 357 (2020).
37. Performance characteristics of the High Sensitivity DNA kit for the Agilent 2100 Bioanalyzer System. (Agilent: 2016).at <https://www.agilent.com/cs/library/technicaloverviews/public/5990-4417EN.pdf>
38. Miotke, L., Lau, B. T., Rumma, R. T. & Ji, H. P. High Sensitivity Detection and Quantitation of DNA Copy Number and Single Nucleotide Variants with Single Color Droplet Digital PCR. Analytical Chemistry 86, 2618-2624 (2014).
39. Tannock, I. F. & Hickman, J. A. Limits to Personalized Cancer Medicine. N. Engl. J. Med. 375, 1289-1294 (2016).
40. Quan, P.-L., Sauzade, M. & Brouzes, E. dPCR: A Technology Review. Sensors 18, 1271 (2018).
41. Luna Universal qPCR Master Mix. (2020).at <https://www.neb.com/products/m3003-luna-universal-qpcr-master-mix #Citations & Technical Literature>
42. Krumbholz, M. et al. Large amplicon droplet digital PCR for DNA-based monitoring of pediatric chronic myeloid leukaemia. Journal of Cellular and Molecular Medicine 23, 4955-4961 (2019).
43. Dujardin, A., Wolf, P. D., Lafont, F. & Dupres, V. Automated multi-sample acquisition and analysis using atomic force microscopy for biomedical applications. PLOS ONE 14, e0213853 (2019).
44. McDermott, G. P. et al. Multiplexed Target Detection Using DNA-Binding Dye Chemistry in Droplet Digital PCR. Analytical Chemistry 85, 11619-11627 (2013).
45. Lau, B. T., Wood-Bouwens, C. & Ji, H. P. Robust Multiplexed Clustering and Denoising of Digital PCR Assays by Data Gridding. Analytical Chemistry 89, 11913-11917 (2017).
46. Binnig, G., Quate, C. & Gerber, C. Atomic force microscope. Phys. Rev. Lett. 56, (1986).
47. Dufrêne, Y. F. et al. Imaging modes of atomic force microscopy for application in molecular and cell biology. Nat. Nanotechnol. 12, 295-307 (2017).
48. Lipiec, E., Japaridze, A., Szczerbiski, J., Dietler, G. & Zenobi, R. Preparation of Well-Defined DNA Samples for Reproducible Nanospectroscopic Measurements. Small 12, 4821-4829 (2016).
49. Reed, J. et al. Identifying individual DNA species in a complex mixture by precisely measuring the spacing between nicking restriction enzymes with atomic force microscope. J. R. Soc. Interface 9, 2341-50 (2012).
50. Japaridze, A. et al. Toward an Effective Control of DNA's Submolecular Conformation on a Surface. Macromolecules 49, 643-652 (2016).
51. Bettotti, P. et al. Structure and Properties of DNA Molecules Over The Full Range of Biologically Relevant Supercoiling States. Scientific Reports 8, (2018).
52. Valle, F., Favre, M., Rios, P. D. L., Rosa, A. & Dietler, G. Scaling Exponents and Probability Distributions of DNA End-to-End Distance. Physical Review Letters 95, (2005).
53. Podesta, A. et al. Positively Charged Surfaces Increase the Flexibility of DNA. Biophysical Journal 89, 2558-2563 (2005).
54. Ando, T., Uchihashi, T. & Kodera, N. High-speed AFM and applications to biomolecular systems. Annu. Rev. Biophys. 42, 393-414 (2013).
55. Uchihashi, T., Watanabe, H., Fukuda, S., Shibata, M. & Ando, T. Functional extension of high-speed AFM for wider biological applications. Ultramicroscopy 160, 182-196 (2016).
56. Payton, O. D., Picco, L. & Scott, T. B. High-speed atomic force microscopy for materials science. International Materials Reviews 61, 473-494 (2016).
57. Watts, M. C. et al. Production of phosphorene nanoribbons. Nature 568, 216-220 (2019).
58. Heaps, E. et al. Bringing real-time traceability to high-speed atomic force microscopy. Measurement Science and Technology 31, 074005 (2020).

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method for detecting length polymorphisms in a DNA sample, comprising:
amplifying a target region of the DNA in the sample, wherein the DNA sample is diluted prior to the amplifying step to provide a plurality of amplified samples in which 0 or 1 target DNA strand is amplified in each amplified sample and wherein at least one amplified sample includes 1 target DNA strand;
depositing the plurality of amplified samples onto a surface;
imaging the plurality of amplified samples deposited on the surface using atomic force microscopy (AFM) to determine an amplicon length distribution;
comparing the amplicon length distribution to a corresponding amplicon length distribution obtained from a reference DNA sample that does not contain a length polymorphism in the target region; and
detecting a length polymorphism in the target region of the DNA sample when the amplicon length distribution is distinct from the corresponding amplicon length distribution.

2. The method of claim 1, wherein the amplifying step is performed using a digital polymerase chain reaction (dPCR).

3. The method of claim 1, wherein the DNA sample and the reference DNA sample are combined into a single sample before the amplifying step in a 1:1 ratio.

4. The method of claim 3, further comprising a step of determining a percentage frequency of the length polymorphism in the DNA sample as compared to the reference DNA sample.

5. The method of claim 1, wherein a Bayesian statistical analysis is used to determine whether the amplicon length distribution is distinct from the corresponding amplicon length distribution of the reference DNA sample.

6. The method of claim 1, wherein the AFM is high-speed AFM.

7. The method of claim 1, wherein the amplicon length distribution is determined by a computer-implemented method comprising the steps of:
flattening an AFM image;
thresholding to identify DNA strands;
skeletonizing the thresholded strands;
determining the longest backbone of each skeleton;
measuring the length of the longest backbone; and
applying a quality filter.

8. The method of claim 1, wherein the length polymorphism is an internal tandem duplication.

9. The method of claim 1, wherein the target region is labeled with CRISPR associated protein 9 (Cas9).


SEQUENCE LISTING (3 sequences)

SEQ ID NO: 1 - DNA, 25 bp, Artificial Sequence
  Synthetic oligonucleotide
actgcctattcctaactgactcatc

SEQ ID NO: 2 - DNA, 23 bp, Artificial Sequence
  Synthetic oligonucleotide
ctttcagcattttgacggcaacc

SEQ ID NO: 3 - DNA, 29 bp, Artificial Sequence
  Synthetic probe
  n (1)..(1): fluorescein
  misc_feature (1)..(1): n is a, c, g, or t
  n (11)..(11): ZEN quencher
  misc_feature (11)..(11): n is a, c, g, or t
  n (29)..(29): Iowa black fluorescence quencher
  misc_feature (29)..(29): n is a, c, g, or t
ncagggaaggntactaggatcaggtgctn